(19) United States
(12) Patent Application Publication
(10) Pub. No.: US 2018/0002662 A1
(43) Pub. Date: Jan. 4, 2018

(54) PURIFICATION OF GERM STEM CELLS BY TARGETING MRP9

(71) Applicant: President and Fellows of Harvard College, Cambridge, MA (US)

(72) Inventors: Michael B. Schultz, Boston, MA (US); Michael S. Bonkowski, Cambridge, MA (US); David A. Sinclair, Chestnut Hill, MA (US)

(73) Assignee: President and Fellows of Harvard College, Cambridge, MA (US)

(21) Appl. No.: 15/543,376

(22) PCT Filed: Jan. 12, 2016
(86) PCT No.: PCT/US2016/012964

§ 371 Date: Jul. 13, 2017

Related U.S. Application Data
(60) Provisional application No. 62/102,820, filed on Jan. 13, 2015.

(51) Int. Cl. C12N 5/075; C07K 16/18; G01R 33/12; G01N 15/14; G01N 21/64

(52) U.S. Cl. CPC C12N 5/0609; C07K 16/18; G01N 2021/6439; G01N 2015/149; G01N 15/1459; C07K 2317/34; C07K 2317/33; G01R 33/1276; G01N 21/6428

(57) ABSTRACT

Provided herein are methods and compositions for the purification and detection of germ stem cells (e.g., oogonial stem cells) based on expression of MRP9.

### BACKGROUND

For many years it was believed that all female mammals were born with all of the oocytes they would ever possess. Over the past decade, however, mounting evidence has arisen of the existence of cells able to maintain oocyte production in the ovary. Such cells have come to be referred to as oogonial stem cells (OSCs, also referred to as Egg Precursor cells, or EggPCs). It has recently been reported that OSCs can be isolated using a cell-sorting approach based on the expression of the protein DEAD box polypeptide 4 (DDX4).

The ability to effectively isolate OSCs is critical for the development and practice of many potential fertility-preservation and gene therapy applications. The current method used for the purification of OSCs, based on the use an antibody raised against the OSC-expressed protein DDX4, has proven problematic and difficult to reproduce. Consequently, the efficacy of this purification process for the isolation of OSCs has been called into question (e.g., Zhang et al., Proc. Natl. Acad. Sci. USA 109:12580-12585 (2012), Oatley and Hunt Biology of Reproduction 86:1-2 (2012)). Thus, there is great need for improved compositions and methods for the purification of oogonial stem cells.

### SUMMARY

Provided herein are methods and compositions for the purification and detection of germ stem cells (GSCs), such as oogonial stem cells (OSCs) or spermatogonial stem cell (SSCs).

In certain aspects, provided herein is a method for purifying a GSC based on its expression of Multidrug Resistance-Associated Protein 9 (MRP9). In some embodiments the method comprises contacting a sample comprising the GSC with an anti-MRP9 antibody that specifically binds to MRP9. In some embodiments, the anti-MRP9 antibody does not specifically bind to DDX4. In some embodiments, the method comprises incubating the sample under conditions such that the anti-MRP9 antibody forms a complex with an MRP9 protein expressed on the surface of the GSC. In some embodiments, the method comprises the step of separating the GSC from other material present in the sample. In some embodiments of the methods described herein, the GSC is separated from other material present in the sample by fluorescent activated cell sorting (FACS) (e.g., in embodiments wherein the anti-MRP9 antibody is linked to a fluorescent label or contacted with a secondary antibody that is linked to a fluorescent label). In some embodiments, the GSC is separated from other material present in the sample by magnetic activated cell sorting (MACS) (e.g., in embodiments wherein the anti-MRP9 antibody is linked to a paramagnetic particle or contacted with a secondary antibody that is linked to a paramagnetic particle). In some embodiments, the GSC is separated from other material present in the sample by adhesion-based cell sorting (e.g., in embodiments wherein the anti-MRP9 antibody is immobilized on a solid support or contacted with a secondary antibody that is immobilized on a solid support).

In some embodiments, the method includes contacting the sample with one or more additional GSC-specific antibodies and incubating the sample under conditions such that the additional anti-GSC specific antibodise form a complex with their target proteinon the surface of the GSC. In some embodiments, the additional GSC-specific antibody is an anti-DDX4 antibody. In some embodiments, the additional GSC-specific antibody is an anti-Fragilis antibody. In some embodiments, the additional GSC-specific antibodies include both an anti-DDX4 antibody and an anti-Fragilis antibody.

In certain aspects, provided herein is a method for generating a culture of GSCs. In some embodiments, the method comprises purifying a GSC based on its expression of MRP9 according to the methods described herein and then culturing the GSC.

In some embodiments, provided herein is a method for generating GSC mitochondria (e.g., OSC mitochondria), the method comprising purifying a GSC based on its expression of MRP9 according to the methods described herein and then isolating the mitochondria from the purified GSCs. In some embodiments, the isolated mitochondria are used in an autologous germline mitochondrial transfer-IVF (AUGMENT-IVF) procedure, for example, as described in Tilly and Sinclair Cell Metabolism 17:838-850 (2013) which is hereby incorporated by reference in its entirety. Exemplary methods for isolating mitochondria and transferring such mitochondria into oocytes for the performance of a AUGMENT-IVF procedure are provided in, for example, U.S. Pat. No. 8,642,329, which is hereby incorporated by reference in its entirety.

In certain aspects, provided herein is a method for detecting a GSC based on its expression of MRP9. In some embodiments, the method includes the step of contacting a sample comprising the GSC with an anti-MRP9 antibody that specifically binds to MRP9. In some embodiments, the antibody does not specifically bind to DDX4. In some embodiments, the method includes the step of incubating the sample under conditions such that the anti-MRP9 antibody forms a complex with an MRP9 protein expressed on the surface of the GSC. In some embodiments, the method includes the step of detecting the GSC by detecting the complex. In some embodiments, the GSC is detected by fluorescence microscopy or FACS. In some embodiments, the method further comprises purifying the detected GSC from other material present in the sample.

In some embodiments of the methods described herein, the GSC is a OSC. In some embodiments, the sample is an ovarian tissue sample. In some embodiments, the method further comprises the step of obtaining the ovarian tissue sample from a subject (e.g., a human subject). In some embodiments, the GSC is a SSC. In some embodiments, the GSC is a mammalian GSC. In some embodiments, the GSC is a human GSC.

In some embodiments of the methods described herein, the anti-MRP9 antibody is monoclonal. In some embodiments, the anti-MRP9 antibody is polyclonal. In some embodiments, the anti-MRP9 antibody specifically binds to an extracellular region of MRP9. In some embodiments, the anti-MRP9 antibody does not specifically bind to an epitope of MRP9 having a sequence of APNPVDD. In some embodiments, the anti-MRP9 antibody specifically binds to an extracellular region of MRP9 having sequence selected from the group consisting of SEQ ID NOs 5-20. In some embodiments, the anti-MRP9 antibody specifically bind to an extracellular region of MRP9 having sequence selected from the group consisting of SEQ ID NOs 6-12. In some embodiments, the anti-MRP9 antibody, the one or more additional anti-GSC antibodies and/or the secondary antibody is linked to a detectable label (e.g., a fluorescent moiety, a radioactive moiety, a paramagnetic moiety, a luminescent moiety and/or a colorimetric moiety). In some embodiments, the anti-MRP9 antibody, the one or more additional anti-GSC antibodies and/or the secondary antibody is immobilized on a solid support.

In some embodiments of the methods described herein, the method further comprises the steps of contacting the sample with a secondary antibody linked to a detectable label that specifically binds to the anti-MRP9 antibody and incubating the sample under conditions such that the secondary antibody forms a complex with the anti-MRP9 antibody. In some embodiments of the methods described herein, the method further comprises the steps of contacting the sample with a secondary antibody linked to a solid support that specifically binds to the anti-MRP9 antibody and incubating the sample under conditions such that the secondary antibody forms a complex with the anti-MRP9 antibody.

In some embodiments of the methods described herein, the method further comprises contaicting the sample with a detectable label that distinguishes live cells from dead cells. In certain embodiments, the detectable label that distinguishes live cells from dead cells is 4′,6-doa,odome-2-phenylindole (DAPI), propidium iodide (PI), 7 Amino-Actinomycin D (7-AAD), TO-PRO-3, and/or a Calcein Dye (e.g., Calcein AM, Calcein Violet AM, Calcein Blue AM).

### BRIEF DESCRIPTION OF FIGURES

FIG. 1 shows that a DDX4 antibody (Abcam 13840) does not bind DDX4 on live cells. Panel (a) depicts a model of antibody binding in live compared to fixed and permeabilized cells. Panel (b) depicts a western blot of DDX4 knockdowns with the DDX4 antibody. Panel (c) depicts the analysis of DDX4 antibody affinity by flow cytometry.

FIG. 2 shows that a DDX4 antibody (Abcam 13840) binds to MRP9 on live cells. Panel (a) depicts the quantification of DDX4 antibody affinity against OSCs treated with siScrambled, siMRP9 #2, or siMRP9#3, as determined by flow cytometry. Panel (b) is a schematic depiction of the amino acid sequence of MRP9, showing the epitopes of the DDX4 and MRP9 antibodies. Panel (c) depicts a western blot of MRP9 knockdowns with the MRP9 antibody.

FIG. 3 depicts a human MRP9 mRNA sequence.

FIG. 4 depicts a human MRP9 amino acid sequence with the putative extracellular sequences indicated in bold.

FIG. 5 depicts a mouse MRP9 mRNA sequence.

FIG. 6 depicts a mouse MRP9 amino acid sequence with the putative extracellular sequences indicated in bold.

FIG. 7 shows the results of a western blot demonstrating that DDX4 is not expressed on the cell surface of OSCs, while MRP9 is present in the cytosol and on the membrane of OSCs.

### DETAILED DESCRIPTION

### General

Provided herein are methods and compositions for the purification and/or detection of germ stem cells (GSCs) such as oogonial stem cells (OSCs) or spermatogonial stem cells (SSCs) based on expression of Multidrug Resistance-Associated Protein 9 (MRP9).

Current methods for isolating GSCs, and in particular OSCs, using antibodies raised against the protein DEAD box polypeptide 4 (DDX4), have proven problematic and difficult to reproduce. As described herein, DDX4 is a poor target for the isolation of live GSCs because it is an intracellular protein, and therefore not accessible by antibodies in live cells. As described herein, MRP9, unlike DDX4, is expressed on the surface of OSCs and is therefore a better target for the purification and/or detection of live cells. Thus, as described herein, antibodies that specifically bind to MRP9 and that are either directly or indirectly associated with a detectable label can be used to detect and/or purify MRP9 expressing GSCs using a wide range of methodology, including fluorescent activated cell sorting (FACS), magnetic activated cell sorting (MACS), adhesion-based cell sorting and fluorescence microscopy.

### Definitions

In order that the present invention may be more readily understood, certain terms are first defined. Additional definitions are set forth throughout the detailed description.

The articles “a” and “an” are used herein to refer to one or to more than one (i.e., to at least one) of the grammatical object of the article. By way of example, “an element” means one element or more than one element.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments.

The terms “antigen binding fragment” and “antigen-binding portion” of an antibody, as used herein, refers to one or more fragments of an antibody that retain the ability to bind to an antigen. Examples of binding fragments encompassed within the term “antigen-binding fragment” of an antibody include Fab, Fab′, F(ab′)₂, Fv, scFv, disulfide linked Fv, Fd, diabodies, single-chain antibodies, NANOBODIES®, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

The term “binding” refers to an association, which may be a stable association, between two molecules, for example, between a polypeptide and an antibody, due to, for example, electrostatic, hydrophobic, ionic and/or hydrogen-bond interactions under physiological conditions.

The terms “CDR”, and its plural “CDRs”, refer to a complementarity determining region (CDR) of an antibody or antibody fragment, which determine the binding character of an antibody or antibody fragment. In most instances, three CDRs are present in a light chain variable region (CDRL1, CDRL2 and CDRL3) and three CDRs are present in a heavy chain variable region (CDRH1, CDRH2 and CDRH3). CDRs contribute to the functional activity of an antibody molecule and are separated by amino acid sequences that comprise scaffolding or framework regions. Among the various CDRs, the CDR3 sequences, and particularly CDRH3, are the most diverse and therefore have the strongest contribution to antibody specificity. There are at least two techniques for determining CDRs: (1) an approach based on cross-species sequence variability (i.e., Kabat et al., Sequences of Proteins of Immunological Interest (National Institute of Health, Bethesda, Md. (1987), incorporated by reference in its entirety); and (2) an approach based on crystallographic studies of antigen-antibody complexes (Chothia et al., Nature, 342:877 (1989), incorporated by reference in its entirety).

The term “epitope” means a protein determinant capable of specific binding to an antibody. Epitopes usually consist of chemically active surface groupings of molecules such as amino acids or sugar side chains. Certain epitopes can be defined by a particular sequence of amino acids to which an antibody is capable of binding. The term “extracellular epitope” refers to an epitope that is located on the outside of a cell's plasma membrane. Exemplary extracellular epitopes of MRP9 are provided in Table 1 and Table 2.

As used herein, the term “monoclonal antibody” refers to an antibody obtained from a population of substantially homogeneous antibodies that specifically bind to the same epitope, i.e., the individual antibodies comprising the population are identical except for possible naturally occurring mutations that may be present in minor amounts. The modifier “monoclonal” indicates the character of the antibody as being obtained from a substantially homogeneous population of antibodies, and is not to be construed as requiring production of the antibody by any particular method.

As used herein, “specific binding” refers to the ability of an antibody to bind to a predetermined antigen or epitope. Typically, an antibody specifically binds to its predetermined antigen or epitope with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (e.g., BSA, casein).

### MRP9

Multidrug Resistance-Associated Protein 9 (MRP9) is also known as ATP-Binding Cassette, Sub-Family C Member 12 (ABCC12). MRP9 is a member of the superfamily of ATP-binding transporters and a member of the MRP subfamily, which is generally involved in multi-drug resistance. Increased expression of MRP9 has been associated with breast cancer. As described herein, MRP9 is specifically expressed on the surface of GSCs and is therefore a useful target for GSC purification.

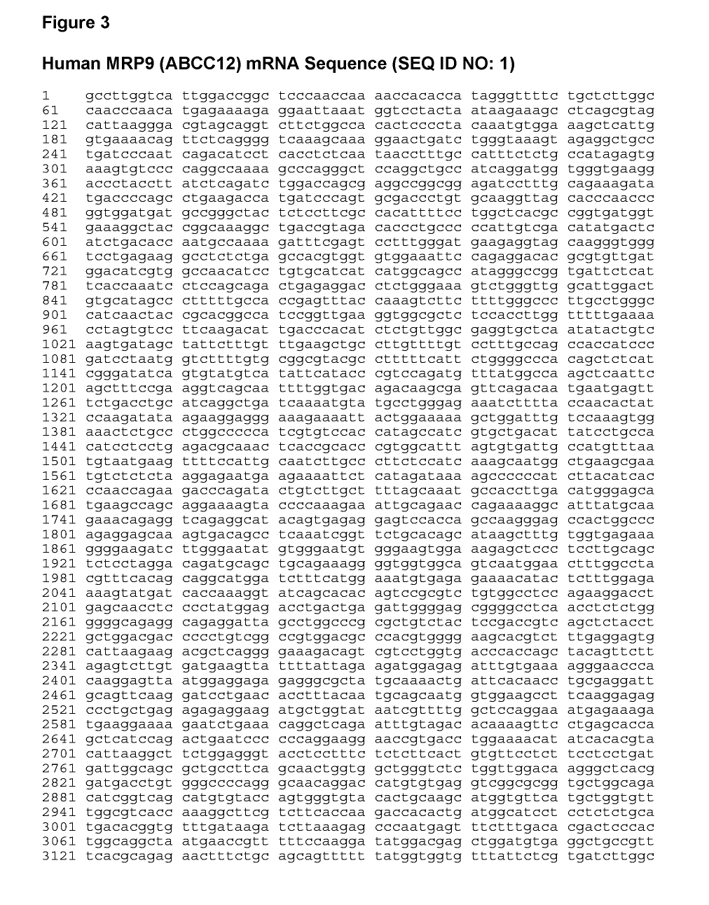

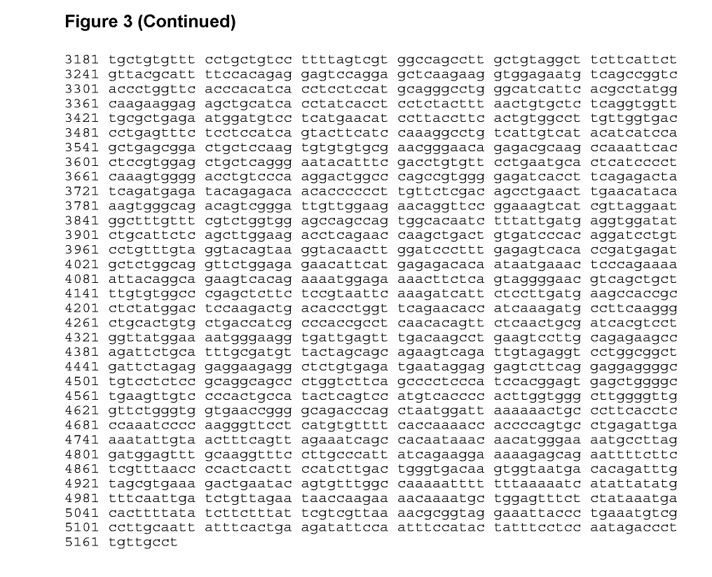

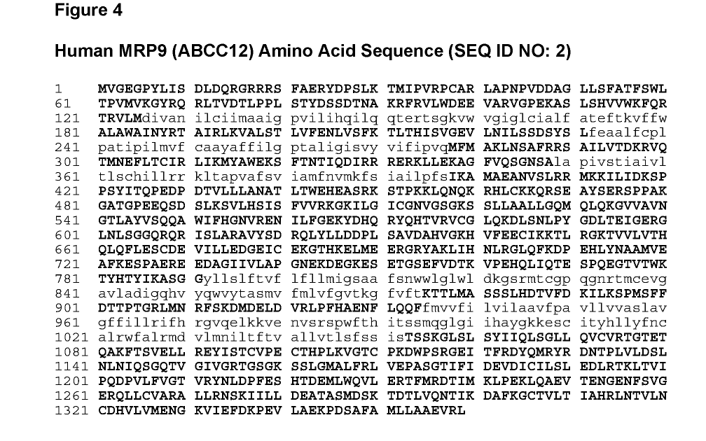

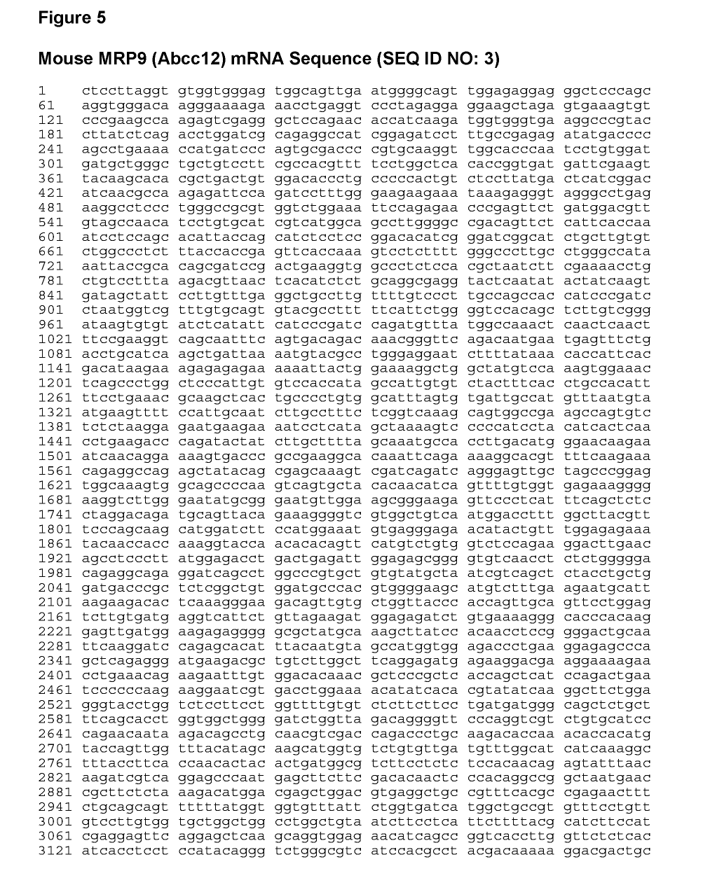

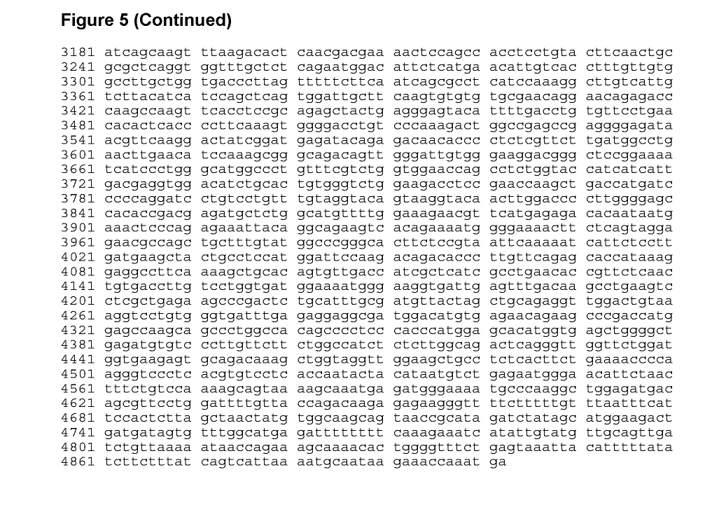

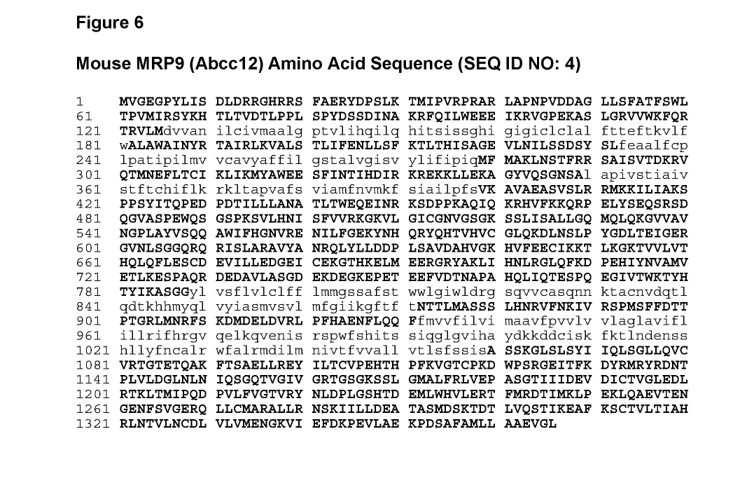

MRP9 is a well conserved protein among vertebrates. The human MRP9 mRNA sequence is provided in FIG. 3 and the human MRP9 protein sequence is provided in FIG. 4 (with putative extracellular sequences capitalized and bold). The mouse MRP9 mRNA sequence is provided in FIG. 5 and the mouse MRP9 protein sequence is provided in FIG. 6 (with putative extracellular sequences capitalized and bold). Sequences for orthologous MRP9 proteins are known in the art and readily available via the NCBI database, including rat (NP_955409.1), zebrafish (XP_009310629.1), chimpanzee (XP_009429022.1), cow (XP_010812911.1), monkey (XP_002802515.1), dog (XP_544420.3), camel (XP_010948502.1), bison (XP_010842202.1), horse (XP_001490812.3), goat (XP_005692126.1), cat (XP_006941550.1) and sheep (XP_004015596.1), each of which is hereby incorporated by reference. Additional orthologues can be readily identified by one of skill in the art by sequence comparison.

### MRP9 Specific Antibodies

In certain embodiments, the compositions and methods provided herein relate to antibodies (including antigen binding fragments thereof) that bind specifically to MRP9. In some embodiments, the MRP9 protein is human (e.g., a human MRP9 protein having an amino acid sequence of SEQ ID NO: 2), mouse (e.g., a mouse MRP9 protein having an amino acid sequence of SEQ ID NO: 4, or an orthologue thereof (e.g., a rat, zebrafish, chimpanzee, cow, monkey, dog, camel, bison, horse, goat, cat or sheep MRP9 protein). In some embodiments, the antibodies bind to a particular epitope of MRP9. In some embodiment the epitope is an extracellular epitope. In some embodiments, the epitope is part of an extracellular domain of MRP9 (i.e. an extracellular epitope). Exemplary extracellular domain sequences of human MRP9 are listed in Table 1, while exemplary mouse extracellular domain sequences of mouse MRP9 are listed in Table 2. In some embodiments, the antibody specifically binds to a polypeptide of SEQ ID NO: 6. In some embodiments, the antibody specifically binds to a polypeptide of SEQ ID NO: 7. In some embodiments, the antibody specifically binds to a polypeptide of SEQ ID NO: 8. In some embodiments, the antibody specifically binds to a polypeptide of SEQ ID NO: 9. In some embodiments, the antibody specifically binds to a polypeptide of SEQ ID NO: 10. In some embodiments, the antibody specifically binds to a polypeptide of SEQ ID NO: 11. In some embodiments, the antibody specifically binds to a polypeptide of SEQ ID NO: 12. In some embodiments, the antibody specifically binds to a polypeptide of SEQ ID NO: 14. In some embodiments, the antibody specifically binds to a polypeptide of SEQ ID NO: 15. In some embodiments, the antibody specifically binds to a polypeptide of SEQ ID NO: 16. In some embodiments, the antibody specifically binds to a polypeptide of SEQ ID NO: 17. In some embodiments, the antibody specifically binds to a polypeptide of SEQ ID NO: 18. In some embodiments, the antibody specifically binds to a polypeptide of SEQ ID NO: 19. In some embodiments, the antibody specifically binds to a polypeptide of SEQ ID NO: 20.

TABLE 1
Exemplary human MRP9 extracellular domain
sequences.

| SEQ | |
|---|---|
| ID | |
| NO: | Extracellular Sequence |
| 5 | MVGEGPYLISDLDQRGRRRSFAERYDPSLKTMIPVRPCARLAPNP |
| | VDDAGLLSFATFSWLTPVMVKGYRQRLTVDTLPPLSTYDSSDTNA |
| | KRFRVLWDEEVARVGPEKASLSHVVWKFQRTRVLM |
| 6 | MVGEGPYLISDLDQRGRRRSFAERYDPSLKTMIPVRPCARL |
| 7 | AGLLSFATFSWLTPVMVKGYRQRLTVDTLPPLSTYDSSDTNAKRF |
| | RVLWDEEVARVGPEKASLSHVVWKFQRTRVLM |
| 8 | ALAWAINYRTAIRLKVALSTLVFENLVSFKTLTHISVGEVLNILS |
| | SDSYS |
| 9 | MFMAKLNSAFRRSAILVTDKRVQTMNEFLTCIRLIKMYAWEKSF |
| | TNTIQDIRRRERKLLEKAGFVQSGNSA |
| 10 | IKAMAEANVSLRRMKKILIDKSPPSYITQPEDPDTVLLLANATLT |
| | WEHEASRKSTPKKLQNQKRHLCKKQRSEAYSERSPPAKGATGPEE |
| | QSDSLKSVLHSISFVVRKGKILGICGNVGSGKSSLLAALLGQMQL |
| | QKGVVAVNGTLAYVSQQAWIFHGNVRENILFGEKYDHQRYQHTVR |
| | VCGLQKDLSNLPYGDLTEIGERGLNLSGGQRQRISLARAVYSDRQ |
| | LYLLDDPLSAVDAHVGKHVFEECIKKTLRGKTVVLVTHQLQFLES |
| | CDEVILLEDGEICEKGTHKELMEERGRYAKLIHNLRGLQFKDPEH |
| | LYNAAMVEAFKESPAEREEDAGIIVLAPGNEKDEGKESETGSEFV |
| | DTKVPEHQLIQTESPQEGTVTWKTYHTYIKASG |
| 11 | KTTLMASSSLHDTVFDKILKSPMSFFDTTPTGRLMNRFSKDMDEL |
| | DVRLPFHAENFLQQF |
| 12 | TSSKGLSLSYIIQLSGLLQVCVRTGTETQAKFTSVELLREYISTC |
| | VPECTHPLKVGTCPKDWPSRGEITFRDYQMRYRDNTPLVLDSLNL |
| | NIQSGQTVGIVGRTGSGKSSLGMALFRLVEPASGTIFIDEVDICI |
| | LSLEDLRTKLTVIPQDPVLFVGTVRYNLDPFESHTDEMWQVLERT |
| | FMRDTIMKLPEKLQAEVTENGENFSVGERQLLCVARALLRNSKII |
| | LLDEATASMDSKTDTLVQNTIKDAFKGCTVLTIAHRLNTVLNCDH |
| | VLVMENGKVIEFDKPEVLAEKPDSAFAMLLAAEVRL |

TABLE 2
Exemplary mouse MRP9 extracellular domain
sequences.

| SEQ | |
|---|---|
| ID | |
| NO: | Extracellular Sequence |
| 13 | MVGEGPYLISDLDRRGHRRSFAERYDPSLKTMIPVRPRARLAPNP |
| | VDDAGLLSFATFSWLTPVMIRSYKHTLTVDTLPPLSPYDSSDINA |
| | KRFQILWEEEIKRVGPEKASLGRVVWKFQRTRVLM |
| 14 | MVGEGPYLISDLDRRGHRRSFAERYDPSLKTMIPVRPRARL |
| 15 | AGLLSFATFSWLTPVMIRSYKHTLTVDTLPPLSPYDSSDINAKRF |
| | QILWEEEIKRVGPEKASLGRVVWKFQRTRVLM |
| 16 | ALAWAINYRTAIRLKVALSTLIFENLLSFKTLTHISAGEVLNILS |
| | SDSYSL |
| 17 | MFMAKLNSTFRRSAISVTDKRVQTMNEFLTCIKLIKMYAWEESFI |
| | NTIHDIRKREKKLLEKAGYVQSGNSA |
| 18 | VKAVAEASVSLRRMKKILIAKSPPSYITQPEDPDTILLLANATLT |
| | WEQEINRKSDPPKAQIQKRHVFKKQRPELYSEQSRSDQGVASPEW |
| | QSGSPKSVLHNISFVVRKGKVLGICGNVGSGKSSLISALLGQMQL |
| | QKGVVAVNGPLAYVSQQAWIFHGNVRENILFGEKYNHQRYQHTV |
| | HVCGLQKDLNSLPYGDLTEIGERGVNLSGGQRQRISLARAVYANR |
| | QLYLLDDPLSAVDAHVGKHVFEECIKKTLKGKTVVLVTHQLQFLE |
| | SCDEVILLEDGEICEKGTHKELMEERGRYAKLIHNLRGLQFKDPE |
| | HIYNVAMVETLKESPAQRDEDAVLASGDEKDEGKEPETEEFVDTN |
| | APAHQLIQTESPQEGIVTWKTYHTYIKASGG |
| 19 | NTTLMASSSLHNRVFNKIVRSPMSFFDTTPTGRLMNRFSKDMDEL |
| | DVRLPFHAENFLQQF |
| 20 | ASSKGLSLSYIIQLSGLLQVCVRTGTETQAKFTSAELLREYILTC |
| | VPEHTHPFKVGTCPKDWPSRGEITFKDYRMRYRDNTPLVLDGLNL |
| | NIQSGQTVGIVGRTGSGKSSLGMALFRLVEPASGTIIIDEVDICT |
| | VGLEDLRTKLTMIPQDPVLFVGTVRYNLDPLGSHTDEMLWHVLER |
| | TFMRDTIMKLPEKLQAEVTENGENFSVGERQLLCMARALLRNSKI |
| | ILLDEATASMDSKTDTLVQSTIKEAFKSCTVLTIAHRLNTVLNCD |
| | LVLVMENGKVIEFDKPEVLAEKPDSAFAMLLAAEVGL |

In some embodiments, the antibody does not specifically bind to a DDX4 protein from the same species as the MRP9 protein to which it binds (e.g., a human DDX4 protein, a mouse DDX4 protein). DDX4 protein sequences are known in the art and readily available through the NCBI database, including human (NP_001136021.1) and mouse (NP_00139357.1), each of which is hereby incorporated by reference. In some embodiments, the antibody does not specifically bind to a MRP9 epitope having an amino acid sequence of APNPVDD.

In some embodiments, the antibody is polyclonal. In some embodiments, the antibody is monoclonal. In some embodiments, the MRP9 antibody can be of any species.

In some embodiments, the antibody is a mouse, rat, sheep, goat, camel, chicken, duck, hamster, guinea pig, dog, monkey, human or synthetic antibody or a combination thereof.

Polyclonal antibodies can be prepared by immunizing a suitable subject (e.g. a mouse, rat, sheep, goat, camel, chicken, duck, hamster, guinea pig, dog, monkey, etc.) with a polypeptide immunogen (e.g., a MRP9 protein (e.g., a protein of SEQ ID NO: 2 or 4) or a fragment thereof (e.g., a polypeptide comprising SEQ ID NO: 5-20). In some embodiments, the polypeptide immunogen comprises an extracellular epitope of MRP9 (e.g., an epitope in SEQ ID NO: 5-20). The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well known techniques, such as protein A chromatography to obtain the IgG fraction.

At an appropriate time after immunization, e.g., when the antibody titers are highest, antibody-producing cells can be obtained from the subject and used to prepare monoclonal antibodies using standard techniques, such as the hybridoma technique originally described by Kohler and Milstein (1975) Nature 256:495-497) (see also Brown et al. (1981) J. Immunol. 127:539-46; Brown et al. (1980)J. Biol. Chem. 255:4980-83; Yeh et al. (1976) Proc. Natl. Acad. Sci. 76:2927-31; and Yeh et al. (1982) Int. J. Cancer 29:269-75), a human B cell hybridoma technique (Kozbor et al. (1983) Immunol. Today 4:72), a EBV-hybridoma technique (Cole et al. (1985) Monoclonal Antibodies and Cancer Therapy, Alan R. Liss, Inc., pp. 77-96) or a trioma techniques. The technology for producing monoclonal antibody hybridomas is well known (see generally Kenneth, R. H. in Monoclonal Antibodies: A New Dimension In Biological Analyses, Plenum Publishing Corp., New York, New York (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically.

As an alternative to preparing monoclonal antibody-secreting hybridomas, a monoclonal antibody that binds to a target protein described herein can be obtained by screening a recombinant combinatorial immunoglobulin library (e.g., an antibody phage display library or an antibody yeast display library) with the appropriate polypeptide such as a MRP9 protein (e.g., a protein of SEQ ID NO: 2 or 4) or a fragment thereof (e.g., a polypeptide comprising SEQ ID NO: 5-20), to thereby isolate immunoglobulin library members that bind the polypeptide.

Additionally, recombinant antibodies specific for a target protein provided herein and/or an extracellular epitope of a target protein provided herein, such as chimeric or antibodies, can be made using standard recombinant DNA techniques. Such chimeric antibodies can be produced by recombinant DNA techniques known in the art, for example using methods described in U.S. Pat. No. 4,816,567; U.S. Pat. No. 5,565,332; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. 5,225,539; Jones et al. (1986) Nature 321:552-525; Verhoeyan et al. (1988) Science 239:1534; and Beidler et al. (1988) J. Immunol. 141:4053-4060.

Human monoclonal antibodies specific for a target protein provided herein and/or an extracellular epitope of a target protein provided herein can be generated using transgenic or transchromosomal mice carrying parts of the human immune system rather than the mouse system. For example, “HuMAb mice” which contain a human immunoglobulin gene miniloci that encodes unrearranged human heavy (μ and γ) and κ light chain immunoglobulin sequences, together with targeted mutations that inactivate the endogenous μ and κ chain loci (Lonberg, N. et al. (1994) Nature 368(6474): 856 859). Accordingly, the mice exhibit reduced expression of mouse IgM or κ, and in response to immunization, the introduced human heavy and light chain transgenes undergo class switching and somatic mutation to generate high affinity human IgGκ monoclonal antibodies (Lonberg, N. et al. (1994), supra; reviewed in Lonberg, N. (1994) Handbook of Experimental Pharmacology 113:49 101; Lonberg, N. and Huszar, D. (1995) Intern. Rev. Immunol. Vol. 13: 65 93, and Harding, F. and Lonberg, N. (1995) Ann. N. Y Acad. Sci 764:536 546). The preparation of HuMAb mice is described in Taylor, L. et al. (1992) Nucleic Acids Research 20:6287 6295; Chen, J. et al. (1993) International Immunology 5: 647 656; Tuaillon et al. (1993) Proc. Natl. Acad. Sci USA 90:3720 3724; Choi et al. (1993) Nature Genetics 4:117 123; Chen, J. et al. (1993) EMBO J. 12: 821 830; Tuaillon et al. (1994) J. Immunol. 152:2912 2920; Lonberg et al., (1994) Nature 368(6474): 856 859; Lonberg, N. (1994) Handbook of Experimental Pharmacology 113:49 101; Taylor, L. et al. (1994) International Immunology 6: 579 591; Lonberg, N. and Huszar, D. (1995) Intern. Rev. Immunol. Vol. 13: 65 93; Harding, F. and Lonberg, N. (1995) Ann. N.Y. Acad. Sci 764:536 546; Fishwild, D. et al. (1996) Nature Biotechnology 14: 845 851. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807.

In certain embodiments, the antibodies provided herein are able to bind to MRP9 protein and/or a portion of MRP9 listed in Table 1 or Table 2 (e.g., an extracellular domain) with a dissociation constant of no greater than 10⁻⁶, 10⁻⁷, 10⁻⁸ or 10⁻⁹M. In some embodiments, the antibodies provided herein do not bind to DDX4 with a dissociation constant of less than 10⁻⁶, 10⁻⁷, 10⁻⁸ or 10⁻⁹M. Standard assays to evaluate the binding ability of the antibodies are known in the art, including for example, ELISAs, Western blots and RIAs. The binding kinetics (e.g., binding affinity) of the antibodies also can be assessed by standard assays known in the art, such as by Biacore analysis.

In some embodiments, antibodies described herein is linked to a detectable label or a solid support. Examples of detectable labels include fluorescent moieties, radioactive moieties, paramagnetic moieties, luminescent moieties and/or colorimetric moieties. In some embodiments, the antibodies described herein are linked to a fluorescent moiety.

Examples of fluorescent moieties include, but are not limited to, Allophycocyanin, Fluorescein, Phycoerythrin, Peridinin-chlorophyll protein complex, Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 568, Alexa Fluor 594, Alexa Fluor 633, Alexa Fluor 635, Alexa Fluor 647, Alexa Fluor 660, Alexa Fluor 680, Alexa Fluor 700, Alexa Fluor 750, Alexa Fluor 790, EGFP, mPlum, mCherry, mOrange, mKO, EYFP, mCitrine, Venus, YPet, Emerald, Cerulean and CyPet.

### Isolation and Detection of Germline Stem Cells

In some embodiments, the methods provided herein include the step of purifying and/or detecting a population of GSCs, such as OSCs or SSCs (e.g., isolating GSCs from a sample and/or detecting GSCs in a sample). In some embodiments, the sample (and the GSCs contained therein) can be derived from any animal. In some embodiments, the sample is a human sample. In some embodiments, the sample is derived from a non-human animal. In some embodiments, the animal is a mammal. In some embodiments, the animal is a non-human mammal. In some embodiments the mammal is a domesticated mammal (e.g., a cow, a pig, a horse, a donkey, a goat, a camel, a cat, a dog, a guinea pig, a rat, a mouse, a sheep, a zebu, a water buffalo, a yak, a llama, an alpaca, a ferret, a rabbit, a caribou, a reindeer). In some embodiments, the methods provided herein include the step of obtaining the sample from the animal.

In some embodiments, the sample is a tissue sample. In some embodiments, the GSCs are OSCs. In some embodiments, the OSCs are isolated from the ovarian tissue of the animal (i.e., from an ovarian tissue sample). In some embodiments, the GSCs are SSCs. In some embodiments, SSCs are isolated from testes tissue (i.e., from a testes tissue sample).

In some embodiments, any technique known in the art and/or described herein can be used to purify and/or detect the GSCs based or MRP9 expression. In some embodiments, the GSCs are contacted with an anti-MRP9 antibody (e.g., an anti-MRP9 specific antibody described herein) under conditions such that the anti-MRP9 ant-MRP9 antibody forms a complex with MRP9 expressed on the surface of the GSCs. In some embodiments, the cells are washed (e.g., by centrifugation) such that unbound antibody is removed from the sample. In some embodiments, GSCs are then purified and/or detected by purifying or detecting cells to which the anti-MRP9 antibody has bound.

In some embodiments, the anti-MRP9 antibody is either directly linked to a detectable label or is indirectly associated with a detectable label (e.g., through a secondary antibody or protein that specifically binds to the anti-MRP9 antibody) and the GSCs are purified and/or detected by targeting cells to which the detectable label is bound. For example, in some embodiments, the detectable label is a fluorescent label and the GSC is purified and/or detected by fluorescent activated cell sorting (FACS) or fluorescence microscopy. In some embodiments, the detectable label is a paramagnetic particle and the GSC is purified and/or detected by magnetic activated cell sorting (MACS).

In some embodiments, the anti-MRP9 antibody is either directly immobilized on a solid support or is indirectly immobilized with a solid support (e.g., through a secondary antibody or protein that specifically binds to the anti-MRP9 antibody) and the GSCs are purified by separating the cells bound to the solid support from other material present in the sample using adhesion based cell sorting. In some embodiments, the protein that specifically binds to the anti-MRP9 antibody is Protein A, Protein C and/or Protein G.

In some embodiments, GSCs purified according to the methods described herein are then grown in culture. Methods for growing GSCs in culture are described, for example, in Zou et al., Nat. Cell. Biol. 11:631-636 (2009), Woods and Tilly, Nature Protocols 8:966-988 (2013), White et al., Nature Medicine 18:413-421 (2012), Kanatsu-Shinohara et al., Biol. Reprod. 69:612-616 (2003) and Kanatsu-Shinohara et al., Biol. Reprod. 72:985-991 (2005), each of which is hereby incorporated by reference in its entirety.

### EXAMPLES

### Example 1

### MRP9 is a Marker for Isolating Oogonial Stem Cells

To determine whether a DDX4 antibody (Abcam 13840) commonly used in the purification of oogonial stem cells (OSCs) binds to DDX4 on live cells, DDX4 expression was inhibited in OSCs using shRNA and anti-DDX4 antibody binding was determined.

OSCs were grown in MEM-α GlutaMax (Invitrogen 32561) with 10% (v/v) FBS (Invitrogen 26140), 1 mM sodium pyruvate (Invitrogen 11360), 0.1 mM NEAA (Invitrogen 11140), pen-strep-glutamine (Invitrogen 10378), N-2 Plus supplement (R&D Systems 212-GD-050) , 0.1 mM β-mercaptoethanol, 1000 units/mL LIF (Millipore ESG1106), 10 ng/mL EGF (Invitrogen PHG0314), 1 ng/mL bFGF (Invitrogen 13256), and 40 ng/mL GDNF (R&D Systems 212-GD-010). The cells were kept in a 37° C. incubator under a humidified atmosphere containing 5% CO₂.

Two DDX4 specific shRNAs and a scrambled, control shRNA were used. sh DDX4#1 (TRCN0000103710), sh DDX4#2 (TRCN0000103711), and control shScrambled (TRC1/1.5) lentivirus were produced by co-transfection of 293T cells with plasmids encoding psPAX2 (Addgene plasmid 12260), and pMD2.G (Addgene plasmid 12259) using X-tremeGENE HP (Roche) in accordance with the manufacturer's protocol. Media was changed 24 hour post-transfection and the virus harvested after 48 and 72 hour, filtered and used to infect OSCs in the presence of 4 mg/mL polybrene (Sigma-Aldrich) via spin infection (2500 rpm, 30 min). Selection of resistant colonies was initiated 24 hour later using 2 mg/mL puromycin (Invivogen).

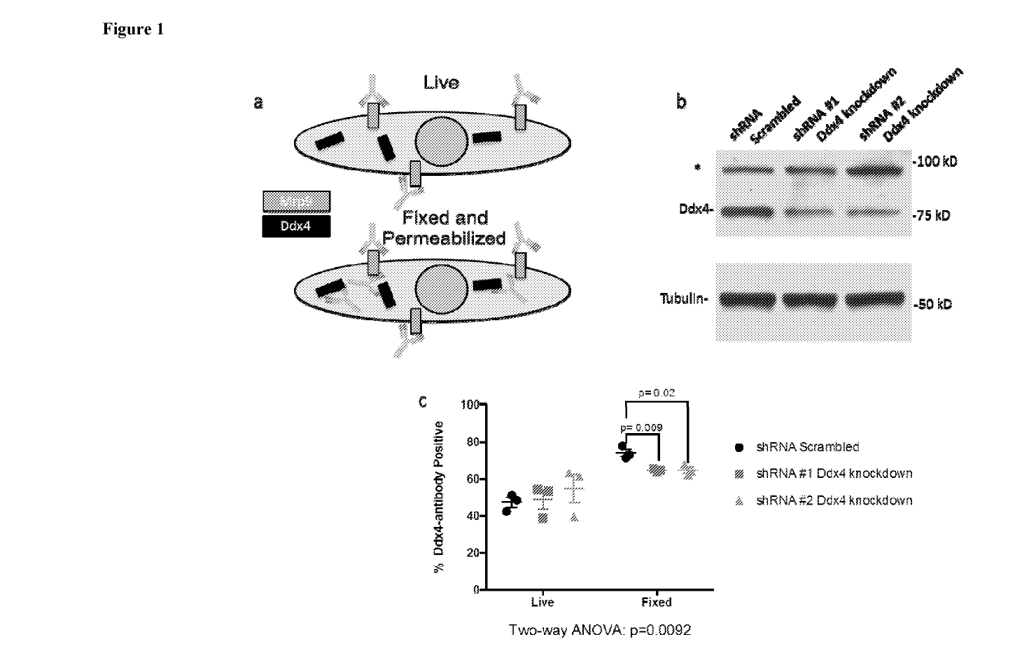

Inhibition of DDX4 protein expression by DDX4 specific shRNA was confirmed by western blot (FIG. 1b). Protein extracts from cultured OSCs were obtained by lysis in ice-cold lysis buffer (150 mM sodium chloride, 50 mM Tris (pH 8.0), 1% NP-40, 0.5% sodium deoxycholate, 0.1% SDS) supplemented with a cocktail of protease and phosphatase inhibitors (Roche). Protein content was determined by the BCA assay (Pierce). Proteins were run on SDS-PAGE under reducing conditions. The separated proteins were then electrophoretically transferred to a polyvinylidene difluoride membrane (Perkin-Elmer). Proteins of interest were revealed with specific antibodies: anti-DDX4 (Abcam 13840) and anti-a-tubulin, overnight at 4° C. The immunostaining was detected using horseradish peroxidase-conjugated anti-rabbit or anti-mouse immunoglobulin for 1 hour at room temperature. Bands were detected using ECL detection reagents (GE Healthcare).

When cells transfected shRNA were analyzed via FACS, cells treated with the DDX4 shRNA showed reduced staining only after they were fixed and permiabilized, but not when stained live and intact (FIG. 1c). Cells were scraped into cold PBS, blocked with normal goat serum (Jackson ImmunoResearch 005-000-121) and stained with anti-DDX4 antibody (Abcam 13840)) and an APC-conjugated secondary antibody (Jackson ImmunoResearch 111-136-144). Sorts were performed on a LSR II Flow Cytometer (BD).

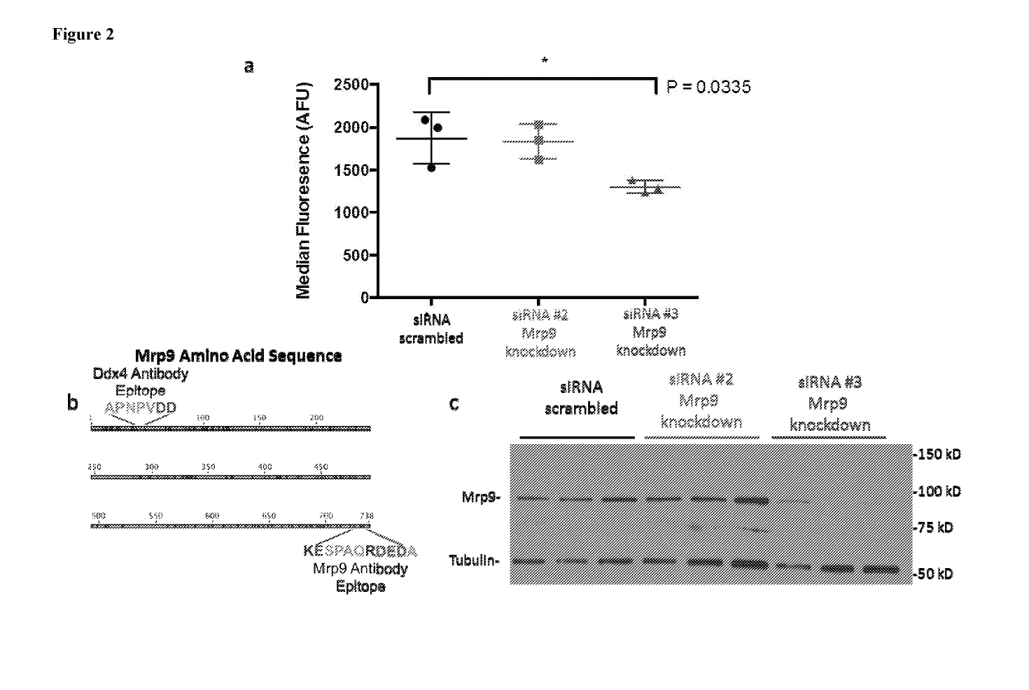

Based on amino acid sequence analysis, it was hypothesized that the anti-DDX4 antibody might be cross-reacting with MRP9 on the surface of the OSCs (FIG. 2b). To determine whether the anti-DDX4 antibody might be binding to MRP9 on live OSCs, siRNA knockdown of MRP9 was employed. siMRP9#1 (D-060119-02, GE Healthcare, AACACCAUUCACGACAUA), siMRP9#2 (D-060119-03, GE Healthcare, CAGGCGAGGUACUCAAUAU), and siScrambled (D-001210-02-05, GE Healthcare) were transfected using Lipofectamine RNAiMAX Reagent (Invitrogen) in accordance with the manufacturer's protocol. Media was changed 24 hour post-transfection and experiments performed 48 hour post-transfection.

Analysis of the siRNA transfected cells by western blot revealed that only one of the two MRP9 siRNAs used reduced MRP9 expression in the cells (FIG. 2c). Protein extracts from cultured OSCs were obtained by lysis in ice-cold lysis buffer (150 mM sodium chloride, 50 mM Tris (pH 8.0), 1% NP-40, 0.5% sodium deoxycholate, 0.1% SDS) supplemented with a cocktail of protease and phosphatase inhibitors (Roche). Protein content was determined by the BCA assay (Pierce). Proteins were run on SDS-PAGE under reducing conditions. The separated proteins were then electrophoretically transferred to a polyvinylidene difluoride membrane (Perkin-Elmer). Proteins of interest were revealed with specific antibodies: anti-MRP9 (Aviva OAAB9500), and anti-a-tubulin, overnight at 4° C. The immunostaining was detected using horseradish peroxidase-conjugated anti-rabbit or anti-mouse immunoglobulin for 1 hour at room temperature. Bands were detected using ECL detection reagents (GE Healthcare).

Staining of live siRNA treated cells with anti-DDX4 antibody and analysis via FACs revealed that cells that had reduced MRP9 expression produced less fluorescent intensity when stained with the anti-DDX4 antibody. Cells were scraped into cold PBS, blocked with normal goat serum (Jackson ImmunoResearch 005-000-121) and stained with anti-DDX4 antibody (Abcam 13840) an APC-conjugated secondary antibody (Jackson ImmunoResearch 111-136-144). Cells were also stained with 1 μg/mL DAPI (Sigma-Aldrich D9542) for exclusion of dead cells. Sorts were performed on a LSR II Flow Cytometer (BD).

### Example 2

### Isolating OSCs based on MRP9 Expression

Ovarian tissue is obtained and minced into a slurry in a small volume of a collagenase/DNase I solution (Type IV Collagenase (Worthington LS004188) and DNase I (Roche 04536282001) in HBSS). The slurry is collected and incubated in an orbital shaker at 37° C. until tissue is digested into a cell suspension. The cell suspension is filtered through a 70-μm nylon mesh cell strainer and the filtrate collected in a 15-ml conical collection tube. The volume of the filtrate is increased to 10 mL by washing the digestion tube with HbSS and filtering further into the collection tube. The strained cell suspension is centrifuged at 300×g at 4° C. for 15 minutes.

Following centrifugation, the liquid is removed and the cell pellet resuspended in blocking buffer (2% bovine serum albumin and 2% normal goat serum in HBSS). About 90-95% of the resuspended cell solution are transferred to the primary sample tube, with the remaining cell solution transferred to separate tubes and used as controls (unstained control, anti-MRP9 antibody only control, secondary antibody only control, DAPI only control). Samples and controls are incubated on ice for 10-15 minutes. HBSS is added to all of the tubes and the anti-MRP9 antibody only control and Sample tubes and cells in these tubes are centrifuged at 300×g at 4° C. for 5 minutes. The supernatant is removed from the anti-MRP9 antibody only control and Sample tubes and the cell pellets are resuspended in anti-MRP9 antibody solution (mouse monoclonal antibody specific for an MRP9 extracellular domain epitope diluted in blocking buffer). The anti-MRP9 antibody only control and Sample tubes are incubated on ice for 10-15 minutes, at which time HBSS is added to the anti-MRP9 antibody only control, secondary antibody only control and Sample tubes, which are centrifuged at 300×g at 4° C. for 5 minutes. The anti-MRP9 antibody only control cell pellet is resuspended in HBSS while the secondary antibody only control and sample cell pellets are resuspended in secondary antibody solution (APC labeled anti-mouse IgG antibody diluted in blocking buffer). The secondary antibody only control and sample tubes are incubated on ice for 10-15 minutes. HBSS is added to the secondary antibody only control and sample tubes and all tubes (controls and sample) are centrifuged at 300×g at 4° C. for 5 minutes. The supernatant is removed from all tubes. The DAPI only control and sample cell pellets are resuspended in FACS buffer (0.5% FBS in HBSS) containing 1 μg/ml DAPI, while the remaining control cell pellets are resuspended in FACS buffer alone. Resuspended cells are filtered into FACS tubes. The control cells are used to calibrate the cell sorter and the MRP9⁺ and DAPI⁻ cells are purified from the sample by FACS.

The above example is for illustration purposes only and one of skill in the art would readily appreciate how such a method could be modified. For example, the primary anti-MRP9 antibody could be directly conjugated to a fluorphore, rendering the secondary antibody unnecessary. Alternatively, the primary antibody or the secondary antibody could be conjugated to a paramagnetic particle and the MRP9 positive cells purified by MACS instead of FACS.

### Example 3

### MRP9, but not DDX4, is Expressed on the Cell Surface of OSCs

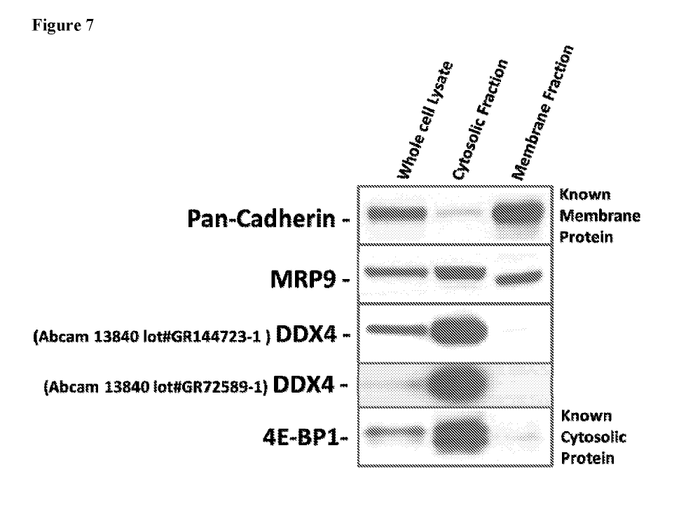

The cellular localization of DDX4 and MRP9 was determined using cultured OSCs. Whole cell homogenates, cytosolic fractions and membrane fractions of cultured OSCs were isolated using gentle lysis and differential specification. Specifically, approximately 8×10⁸ OSCs were raised in OCC culture to 80% confluency. Cells were scraped, washed twice in PBS and pelleted. Whole cells were homogenized and cytosolic versus membrane fractions were purified and prepared for SDS-page and immunoblotting using the Abcam Plasma Membrane Purification Protein Extraction Kit (ab65400). Whole cell lysate and isolated cellular fractions were then run on SDS-page gels under reducing conditions and then electrophoretically transferred to a polyvinylidene difluoride membrane (Perkin-Elmer). The membranes were then probed with antibodies directed towards a control cell membrane protein (E-Cadherin, Cell Signaling #4068S), a control cytosolic protein (4E-BP1, Cell Signaling #9452S), MRP9 (Aviva OAAB9500) and DDX4 (Abcam 13840, lot#s GR72589-1, GR144723-1), overnight at 4° C. The immunostaining was detected using horseradish peroxidase-conjugated anti-rabbit or anti-mouse immunoglobulin for 1 hour at room temperature. Bands were detected using ECL detection reagents (GE Healthcare). As seen in FIG. 7, DDX4 is only present in the cytosolic fraction, while MRP9 is abundantly present in both the membrane and cytosolic fractions

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Those skilled in the art will recognize, or be able to ascertain using no more than routine experimentation, many equivalents to the specific embodiments of the invention described herein. Such equivalents are intended to be encompassed by the following claims.

What is claimed is:

1. A method for purifying a germ stem cell (GSC), the method comprising:
(a) contacting a sample comprising the GSC with an anti-MRP9 antibody that specifically binds to MRP9 and that does not specifically bind to DDX4;
(b) incubating the sample under conditions such that the anti-MRP9 antibody forms a complex with an MRP9 protein expressed on the surface of the GSC; and
(c) separating the GSC from other material present in the sample.

2. The method of claim 1, wherein the GSC is a oogonial stem cell (OSC).

3. The method of claim 2, wherein the sample is an ovarian tissue sample.

4. The method of claim 3, further comprising the step of obtaining the ovarian tissue sample from a subject.

5. The method of claim 1, wherein the GSC is a spermatogonial stem cell (SSC).

6. The method of any one of claims 1 to 5, wherein the GSC is a human GSC.

7. The method of any one of claims 1 to 6, wherein the anti-MRP9 antibody is monoclonal.

8. The method of any one of claims 1 to 6, wherein the anti-MRP9 antibody is polyclonal.

9. The method of any one of claims 1 to 8, wherein the anti-MRP9 antibody specifically binds to an extracellular region of MRP9.

10. The method of any one of claims 1 to 9, wherein the anti-MRP9 antibody does not specifically bind to an epitope having a sequence of APNPVDD.

11. The method of any one of claims 1 to 10, wherein the anti-MRP9 antibody specifically binds to an extracellular region of MRP9 having a sequence selected from the group consisting of SEQ ID NOs 5-20.

12. The method of any one of claims 1 to 10, wherein the anti-MRP9 antibody specifically bind to an extracellular region of MRP9 having a sequence selected from the group consisting of SEQ ID NOs 6-12.

13. The method of any one of claims 1 to 12, wherein the GSC is separated from other material present in the sample in step (c) by fluorescent activated cell sorting (FACS).

14. The method of any one of claims 1 to 12, wherein the GSC is separated from other material present in the sample in step (c) by magnetic activated cell sorting (MACS).

15. The method of any one of claims 1 to 14, wherein the anti-MRP9 antibody is linked to a detectable label.

16. The method of claim 15, wherein the detectable label is a fluorescent label and the GSC is separated from other material present in the sample in step (c) by FACS.

17. The method of claim 15, wherein the detectable label is a paramagnetic particle and the GSC is separated from other material present in the sample in step (c) by MACS.

18. The method of any one of claims 1 to 12, wherein the method further comprises, between steps (b) and (c), the steps of:
(i) contacting the sample with a secondary antibody that specifically binds to the anti-MRP9 antibody, wherein the secondary antibody is linked to a detectable label; and
(ii) incubating the sample under conditions such that the secondary antibody forms a complex with the anti-MRP9 antibody.

19. The method of claim 18, wherein the detectable label is a fluorescent label and the GSC is separated from other material present in the sample in step (c) by FACS.

20. The method of claim 18, wherein the detectable label is a paramagnetic particle and the GSC is separated from other material present in the sample in step (c) by MACS.

21. A method for generating a culture of germ stem cells (GSCs), the method comprising:
(a) contacting a sample comprising the GSCs with anti-MRP9 antibodies that specifically bind to MRP9 and that do not specifically bind to DDX4;
(b) incubating the sample under conditions such that the anti-MRP9 antibodies form complexes with MRP9 proteins expressed on the surface of the GSCs;
(c) separating the GSCs from other material present in the sample; and
(d) culturing the GSCs.

22. The method of claim 21, wherein the GSCs are oogonial stem cells (OSCs).

23. The method of claim 22, wherein the sample is an ovarian tissue sample.

24. The method of claim 23, further comprising the step of obtaining the ovarian tissue sample from a subject.

25. The method of claim 21, wherein the GSCs are a spermatogonial stem cells (SSCs).

26. The method of any one of claims 21 to 25, wherein the GSCs are human GSCs.

27. The method of any one of claims 21 to 26, wherein the anti-MRP9 antibodies are monoclonal.

28. The method of any one of claims 21 to 26, wherein the anti-MRP9 antibodies are polyclonal.

29. The method of any one of claims 21 to 28, wherein the anti-MRP9 antibodies specifically bind to an extracellular region of MRP9.

30. The method of any one of claims 21 to 29, wherein the anti-MRP9 antibodies do not specifically bind to an epitope having a sequence of APNPVDD.

31. The method of any one of claims 21 to 30, wherein the anti-MRP9 antibodies specifically bind to an extracellular region of MRP9 having sequence selected from the group consisting of SEQ ID NOs 5-20.

32. The method of any one of claims 21 to 30, wherein the anti-MRP9 antibodies specifically bind to an extracellular region of MRP9 having a sequence selected from the group consisting of SEQ ID NOs 6-12.

33. The method of any one of claims 21 to 32, wherein the GSCs are separated from other material present in the sample in step (c) by fluorescent activated cell sorting (FACS).

34. The method of any one of claims 21 to 32, wherein the GSCs are separated from other material present in the sample in step (c) by magnetic activated cell sorting (MACS).

35. The method of any one of claims 21 to 34, wherein the anti-MRP9 antibodies are linked to a detectable label.

36. The method of claim 35, wherein the detectable label is a fluorescent label and the GSCs are separated from other material present in the sample in step (c) by FACS.

37. The method of claim 35, wherein the detectable label is a paramagnetic particle and the GSCs are separated from other material present in the sample in step (c) by MACS.

38. The method of any one of claims 21 to 32, wherein the method further comprises, between steps (b) and (c), the steps of:
(i) contacting the sample with secondary antibodies that specifically bind to the anti-MRP9 antibodies, wherein the secondary antibodies are linked to a detectable label; and
(ii) incubating the sample under conditions such that the secondary antibodies form a complex with the anti-MRP9 antibodies.

39. The method of claim 38, wherein the detectable label is a fluorescent label and the GSCs are separated from other material present in the sample in step (c) by FACS.

40. The method of claim 38, wherein the detectable label is a paramagnetic particle and the GSCs are separated from other material present in the sample in step (c) by MACS.

41. A method for detecting a germ stem cell (GSC), the method comprising:
(a) contacting a sample comprising the GSC with an anti-MRP9 antibody that specifically binds to MRP9 and that does not specifically bind to DDX4;
(b) incubating the sample under conditions such that the anti-MRP9 antibody forms a complex with an MRP9 protein expressed on the surface of the GSC; and
(c) detecting the GSC by detecting the complex.

42. The method of claim 41, wherein the GSC is a oogonial stem cell (OSC).

43. The method of claim 42, wherein the sample is an ovarian tissue sample.

44. The method of claim 43, further comprising the step of obtaining the ovarian tissue sample from a subject.

45. The method of claim 41, wherein the GSC is a spermatogonial stem cell (SSC).

46. The method of any one of claims 41 to 45, wherein the GSC is a human GSC.

47. The method of any one of claims 41 to 46, wherein the anti-MRP9 antibody is monoclonal.

48. The method of any one of claims 41 to 46, wherein the anti-MRP9 antibody is polyclonal.

49. The method of any one of claims 41 to 48, wherein the anti-MRP9 antibody specifically binds to an extracellular region of MRP9.

50. The method of any one of claims 41 to 49, wherein the anti-MRP9 antibody does not specifically bind to an epitope having a sequence of APNPVDD.

51. The method of any one of claims 41 to 50, wherein the anti-MRP9 antibody specifically binds to an extracellular region of MRP9 having a sequence selected from the group consisting of SEQ ID NOs 5-20.

52. The method of any one of claims 41 to 50, wherein the anti-MRP9 antibody specifically bind to an extracellular region of MRP9 having a sequence selected from the group consisting of SEQ ID NOs 6-12.

53. The method of any one of claims 41 to 52, wherein the GSC detected in step (c) by fluorescence microscopy.

54. The method of any one of claims 41 to 52, wherein the GSC is detected in step (c) by fluorescence activated cell sorting (FACS).

55. The method of any one of claims 41 to 54, wherein the anti-MRP9 antibody is linked to a detectable label.

56. The method of claim 55, wherein the detectable label is a fluorescent label and the GSC is detected in step (c) by fluorescence microscopy.

57. The method of claim 55, wherein the detectable label is a fluorescent label and the GSC is detected in step (c) by FACS.

58. The method of any one of claims 41 to 52, wherein the method further comprises, between steps (b) and (c), the steps of:
(i) contacting the sample with a secondary antibody that specifically binds to the anti-MRP9 antibody, wherein the secondary antibody is linked to a detectable label; and
(ii) incubating the sample under conditions such that the secondary antibody forms a complex with the anti-MRP9 antibody.

59. The method of claim 58, wherein the detectable label is a fluorescent label and the GSC is detected in step (c) by fluorescence microscopy.

60. The method of claim 58, wherein the detectable label is a fluorescent label and the GSC is detected in step (c) by FACS.

61. The method of any one of claims 41 to 60, further comprising the step of purifying the GSC from other material present in the sample following step (c).
